(19) United States
(12) Patent Application Publication
(10) Pub. No.: US 2005/0085893 A1
(43) Pub. Date: Apr. 21, 2005

(54) Low-profile, endoluminal prosthesis and deployment device

(76) Inventor: Sumit Roy, Oslo (NO)

Correspondence Address: Sumit Roy, Vaekeroveien 106, Oslo, N-0383, NO

(21) Appl. No.: 10/487,818

(22) PCT Filed: Aug. 20, 2002
(86) PCT No.: PCT/NO02/00285

Related U.S. Application Data
(60) Provisional application No. 60/314,138, filed on Aug. 20, 2001.

(51) Int. Cl. A61F 2/06

(52) U.S. Cl. 623/1.13; 623/1.35

(57) ABSTRACT

An implantable prosthesis for placement in hollow tubular organs is described alongwith an instrument for deploying the said prosthesis. On radial compaction, the prosthesis has a low profile, allowing introduction into the body with a deployment instrument of low calibre. The prosthesis has multiple longitudinal struts to provide longitudinal support. The prosthesis may be provided with helically configured members for circumferential support. The deployment instrument includes a retrievable tool to temporarily secure the prosthesis within the body during the implantation procedure.

### RELATED APPLICATION

This patent application claims priority from U.S. Provisional Patent Application Ser. No. 60/314,138, filed on Aug. 20, 2001, the entire disclosure of the application being expressly incorporated herein by reference.

### TECHNICAL FIELD

This invention under consideration concerns a prosthetic device and related instrument for non-surgically treating diseases of tubular organs of the human body, and methods for using the using the said prosthesis for the said purpose.

### BACKGROUND ART

Over the past two decades, treatment of diseases by the transluminal placement of a prosthesis has garnered increasing attention. In the field of vascular disease, this therapeutic modality now represents the intervention of choice for most occlusive lesions. The satisfactory results obtained with this treatment strategy has encouraged its application for the management of lesions such as aneurysms which are characterized by partial or complete loss of structural integrity rather than hindrance to blood flow. Multiple endoluminal grafts have been described for the purpose, only a few of which have survived the rigours of clinical use. Experience with these prostheses has demonstrated that while they do have therapeutic value, all suffer from a common drawback. They are too bulky to be implanted without creating a surgical vascular access, thereby negating one of the major advantages of the transluminal approach. This characteristic also make them difficult to implant in patients with tortuous blood vessels. Another limitation associated with their use is the inability to treat lesions involving the craniocerebral or visceral branches of the aorta. That all the endoluminal grafts in use have the same disadvantage is not a coincidence because all are based on the same underlying design: a flexible non-porous tube braced by an expandable metallic skeleton.

Reducing the metallic skeleton to a single, sturdy metallic collar has been proposed as one way to reduce the bulk of a endovascular graft during introduction (PCT International Application WO 97/48350). While this modification certainly makes for a more streamlined device, it does not eliminate the need for surgically creating a vascular access because the introducer catheter required has an outer diameter of approximately 5 mm (15 Fr). Furthermore, clinical experience indicates that the absence of support along the longitudinal axis of the device is likely to increase the risk of complications associated with its use such as migration (Resch T. et. al. J Vasc Intervent Radiol 1999; 10:257-64). Deployment of the tubular component of the prosthesis and its metallic skeleton in sequence offers the possibility of reducing the calibre of the introducer system necessary for deployment. Two implantable devices based on this concept have been described thus far (U.S. Pat. No. 5,776,186, U.S. Pat. No. 6,015,422). However neither can be compacted to the degree necessary for percutaneous implantation.

Thus there exists a need for a prosthesis for transluminal implantation that has a low enough profile to be introduced into the body by the non-surgical, percutaneous, approach and yet has sufficient longitudinal rigidity to minimise the risk of complications, and sufficient longitudinal flexibility to accommodate geometrical changes that often occur in tubular organs such as the aorta. The prosthesis should preferably not have a metal skeleton with multiple bent struts, which carries with it the risk of the type of structural failure that contributed to the AneuRx endovascular graft being withdrawn from the market (FDA Public Health Notification, Apr. 21, 2001). Likewise the prosthesis should be free of hooks eliminating the chances of severe vascular trauma observed with the Ancure device (FDA Public Health Notification, Apr. 21, 2001). These requirements are fulfilled by the invention under consideration.

### BRIEF DESCRIPTION OF THE DRAWINGS

To facilitate understanding, throughout the description, the adjective “leading” identifies the end or edge of an object, such as a prosthesis or device, that precedes the rest of said object, when said object is being introduced into another object such as the human body.

FIG. 1 is a perspective view of the preferred embodiment of the implantable prosthesis.

FIG. 2 is a perspective view of the bifurcated embodiment of the implantable prosthesis.

FIG. 3 is a perspective view of an alternative embodiment of the implantable prosthesis characterized by a trailing end narrower than the rest of the prosthesis.

FIG. 4 is a perspective view of the bifurcated embodiment of the implantable prosthesis characterized by one distal limb.

FIG. 5 is a perspective view partly in section of the introducer catheter with the dilator in situ.

FIG. 6 is a perspective view partly in section of the introducer catheter with the pusher in situ.

FIG. 7 is a perspective view of the anchor.

FIG. 8 is a perspective view of an alternative embodiment of the implantable prosthesis characterized by a circumferential slit in the graft.

FIG. 9 is a perspective view partly see-through view of an alternative embodiment of the implantable prosthesis provided with an internal helical support.

FIG. 10 is a perspective view partly see-through view of an alternative embodiment of the implantable prosthesis provided with an internal helical support and short supporting struts.

FIG. 11 is a perspective view partly in section of a loading cartridge containing an the alternative embodiment of the prosthesis provided with an internal helical support.

FIG. 12 is a longitudinal sectional view of the anchor within a cannula.

FIG. 13 is a perspective view partly in section of the loading cartridge with the prosthesis and anchor in situ.

FIG. 14 is a perspective view partly in section of the loading cartridge with the prosthesis and anchor and pusher in situ.

FIG. 15 is a perspective view of the trailing end of the loading cartridge.

FIGS. 16 to 21 are longitudinal sectional views illustrating implantation of a prosthesis in an abdominal aorta with an aneurysm according to the invention.

### DISCLOSURE OF THE INVENTION

### DETAILED DESCRIPTION

The invention is made from biocompatible materials. The materials used to make the components for permanent implantation are in addition characterised by long-term dimensional, structural, and configurational stability under cyclic loading.

FIG. 1 illustrates the preferred embodiment of the invention. The primary component of the invention is a uni- or multilamellar tube (tubular graft) 1 with circular or elliptical cross-section, made from a flexible polymer. Multiple linear strips or wires (struts) 2 of a flexible metal or metal alloy or a flexible polymer is bonded to the graft 1, parallel to the longitudinal axis of the graft 1. The struts 2 may be bonded to the inner or outer surface of the graft 1, or sandwiched between two adjacent lamellae. The leading and trailing free edges 3, 4 of the tube may or may not be parallel to each other, or perpendicular to the longitudinal axis of the tube. The modifications to the graft 1, described in this may also be incorporated singly or in various combinations in the remaining embodiments of the invention.

In the second embodiment of the invention, the struts 2 extend beyond the leading edge 3, or the trailing edge 4 of the graft 1, or both the leading and the trailing edges.

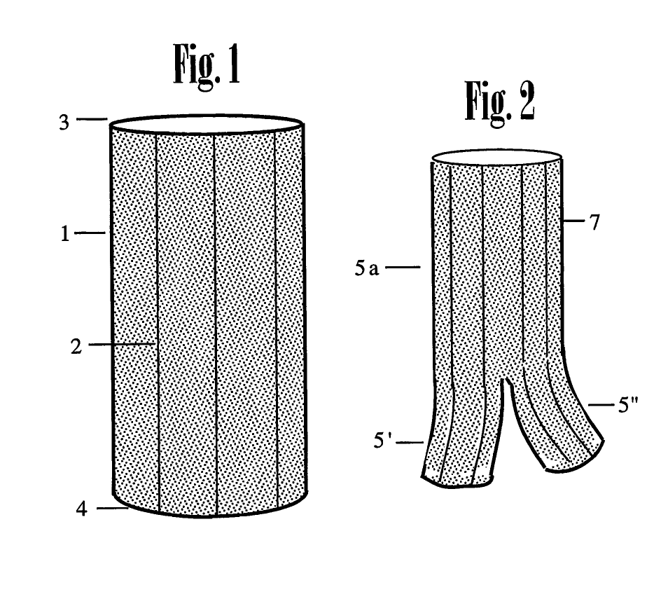

The third embodiment of the invention (aorto-biiliac graft) 5a has a branched configuration with two peripheral limbs (6′, 6″) as illustrated in FIG. 2.

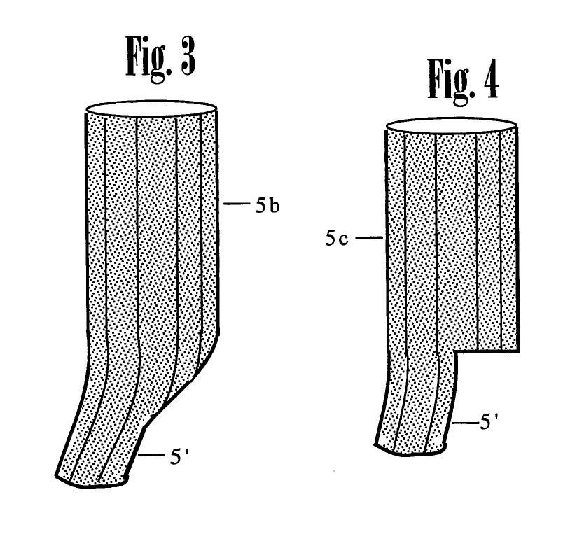

The fourth embodiment of the invention (aorto-iliac graft) 5b has one peripheral limb (6′) as illustrated in FIG. 3.

The fifth embodiment of the invention (aorto-uniiliac graft) 5c has the configuration illustrated in FIG. 4.

The sixth embodiment of the invention (trans-renal aortic graft) is provided with a circumferential slit as illustrated in FIG. 8.

In the seventh embodiment of the invention, the inner surface of the graft 1 is provided with one or more helically configured, reversibly deformable linear members (internal helical support) 2a (FIG. 9). The diameter of the helices is equal to or greater than the inner diameter of the graft 1. Only the leading end of the helical strut 2a is attached to the graft 1, whereby straightening of the helical support will allow radial compaction of the graft. In the eight embodiment of the invention, the struts 2 are made of a resorbable material. In the ninth embodiment of the invention, the struts 2 protrude beyond the leading edge 3 of graft 1 and do not span the length of the graft 1 (FIG. 10). In the tenth embodiment of the invention, one or more helically configured, reversibly deformable linear members are included that are not attached to the graft 1 (external helical support). The diameter of the external helical support is less than or equal to the outer diameter of the graft 1.

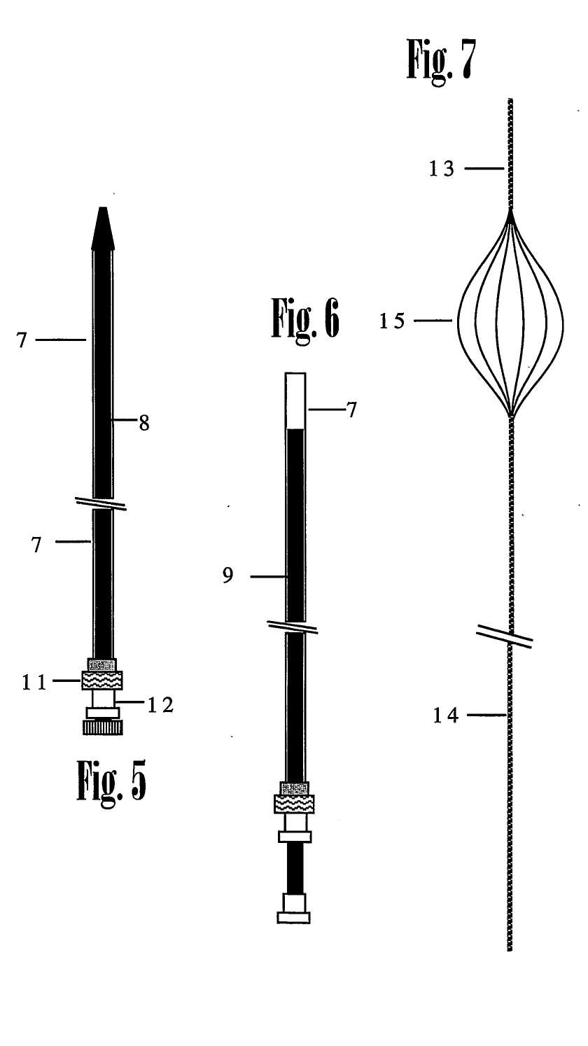

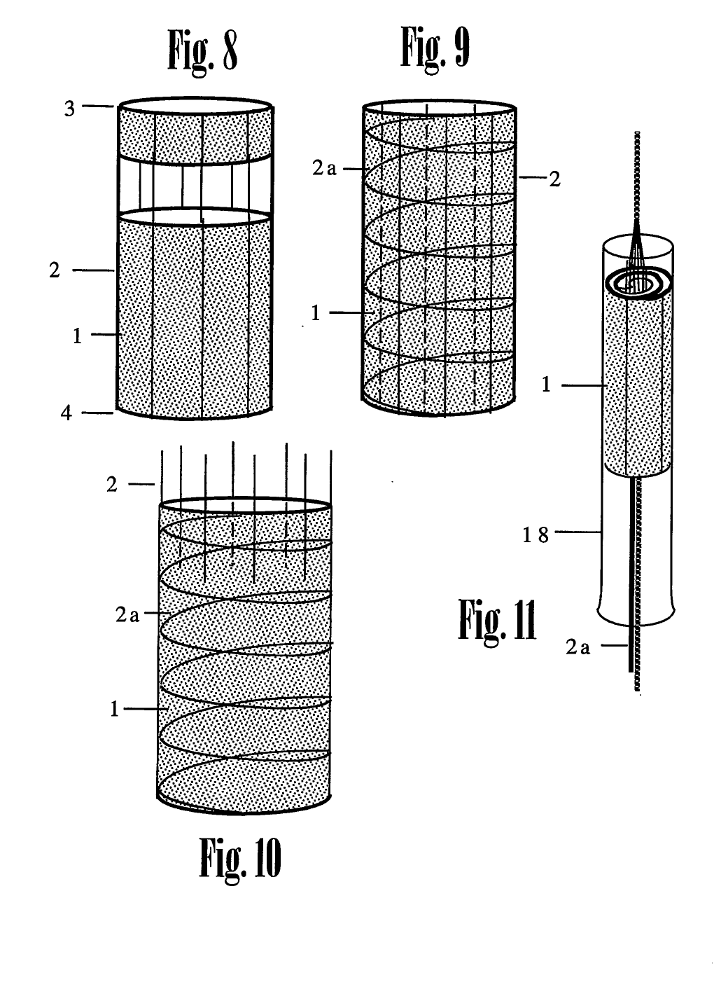

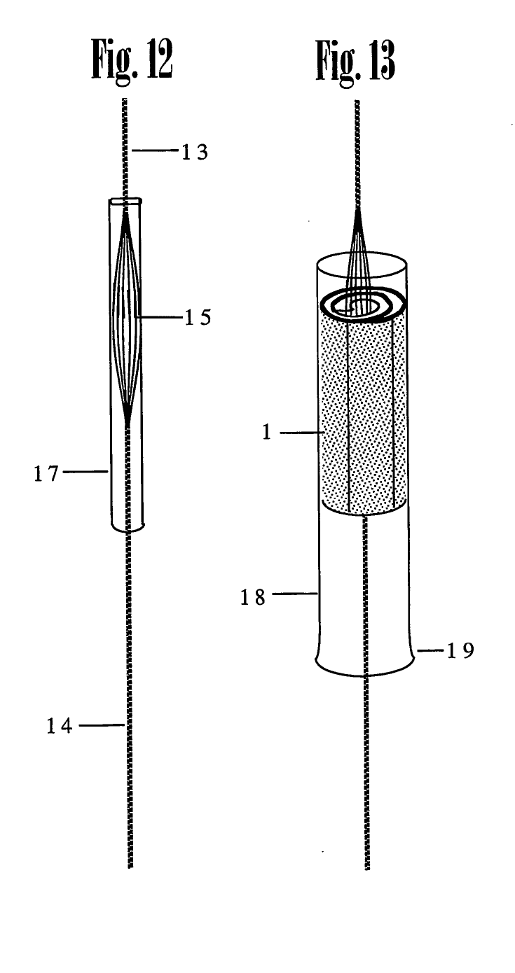

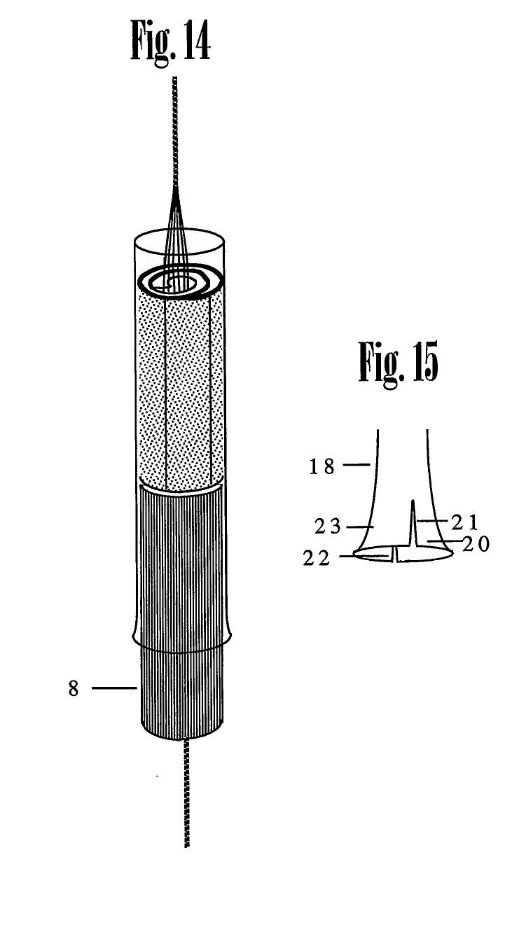

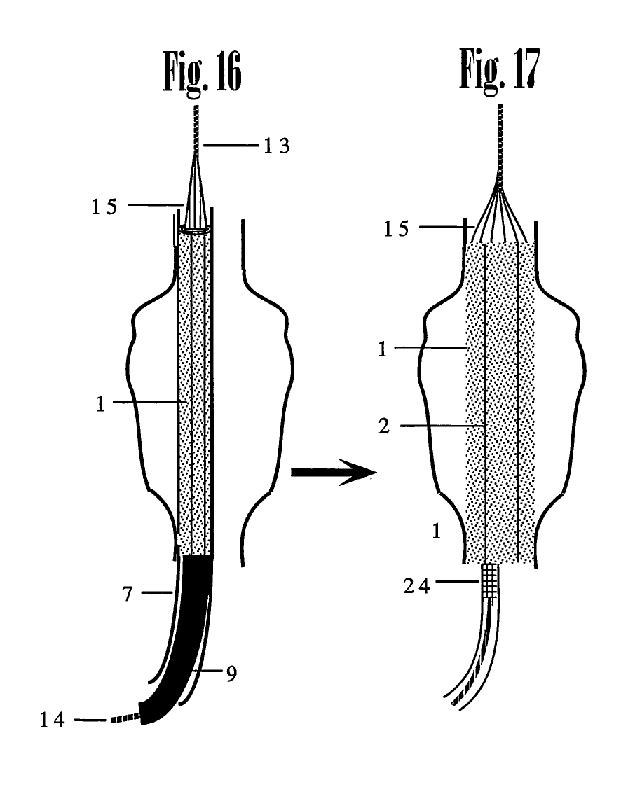

The delivery system to implant the graft comprises a thin wall catheter 7 (deployment catheter), that accommodates a thick-wall catheter with a tapered tip 8 (dilator) (FIG. 5), or a thick-wall catheter with a blunt tip 9 (pusher) (FIG. 6), and a self-expanding retrievable device (anchor) 10 (FIG. 7). The deployment catheter 7 is fitted with a Touhy-Borst valve 11 carrying a female Luer hub 12. The lumen of the deployment catheter 7 communicates with the lumen of the female Luer hub 12, through the Touhy-Borst valve 11. The anchor 10 consists of a leading linear, resilient member 13 (guide) and a trailing linear, resilient member 14 (shaft), that are connected to each other by multiple, outwardly biased, spirally-oriented resilient members with shape memory 15 (basket), that enclose an ovoid-shaped space (FIG. 7). The basket 15 is radially compressible and its long axis is co-linear and co-planer with the guide 13 and the shaft 14.

Use of the Invention

I. Preparation for Implantation:

It is anticipated that this step will be performed at the site of manufacture before the device is sterilised.

The basket is radially compressed and introduced into a cannula 17 (FIG. 12). The graft 1 is tightly rolled around the cannula 17 and a thin-wall polymer tube (loading cartridge) 18 is drawn over the rolled-up graft to prevent it from unravelling (FIG. 13). The cannula 17 is then removed. The pusher is advanced over the shaft 14 of the anchor 10 until its tip abuts the rolled up graft 1 (FIG. 14). A Tuohy-Borst valve is attached to the hub of the dilator 8. The valve is tightened securing the anchor 10 to the dilator 8. The trailing end 19 of the loading cartridge 18 is flared and its free edge has two symmetrically placed slits 20, 21 extending a short distance along the length of the loading cartridge 18, creating two flaps 22,23 (FIG. 15). By applying traction on the flaps 22,23 perpendicular to the longitudinal axis of the loading cartridge 18, the latter can be split into two separate parts.

In case of the seventh, eighth, ninth or tenth embodiments of the invention, the internal helical support 2a is straightened before the graft 1 is rolled around cannula 17, such that the trailing end of the internal helical support protrudes from the trailing end of the graft 1 (FIG. 11).

II. Implantation of Graft:

The implantation procedure for lesions involving the infrarenal aorta and its bifurcation are described. These represent only examples to illustrate some of the envisaged uses of the invention and do not limit in any way the scope of its application as set forth in this provisional patent application. Furthermore the deployment of a single graft per site is described. Multiple grafts may be coaxially deployed using the same or similar procedure, if warranted by the anticipated circumferential stresses at the site of the lesion.

A. Prosthesis Without Helical Support:

(a) Implantation in the Infrarenal Aorta:

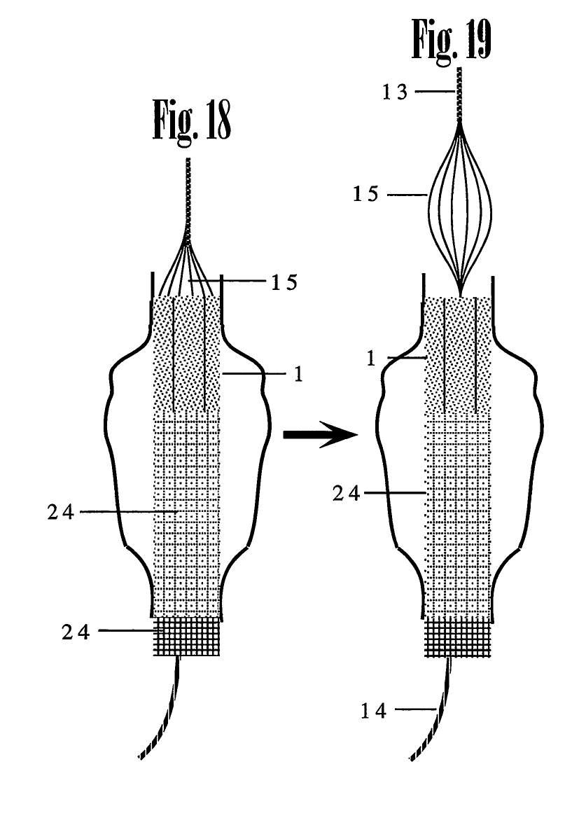

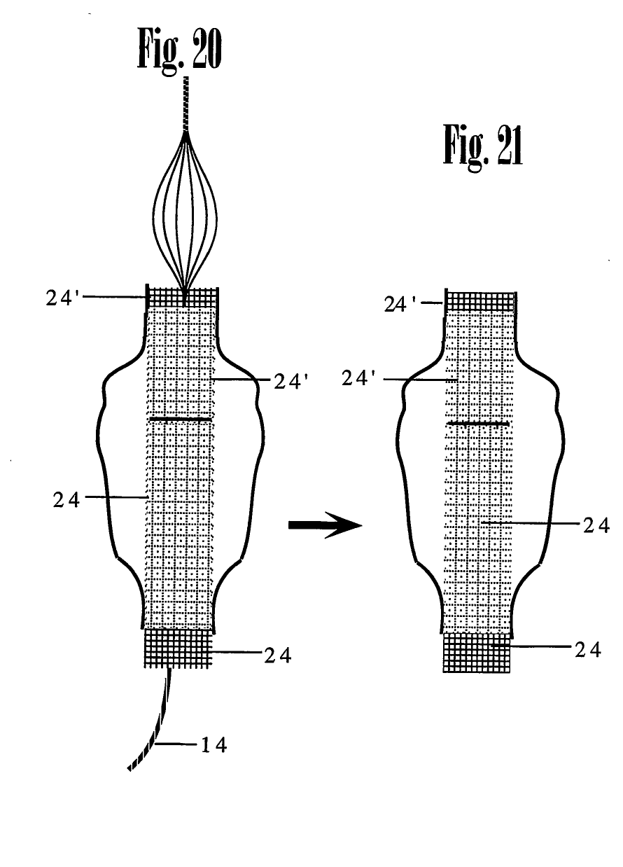

After the anatomy of the lesion has been satisfactorily determined, a guidewire is placed traversing the lesion. The thin-wall catheter 7 carrying its corresponding dilator 8 is introduced coaxially over the guidewire and advanced until it spans the lesion. The dilator 8 is removed. The loading cartridge 18 is introduced into the Luer hub 12 of the thin-wall catheter 7. The Touhy-Borst valve 11 is opened and axial force applied to the pusher 9 to backload the graft 1 into the thin-wall catheter 7. Once the entire graft 1 has passed beyond the haemostatic valve, the loading cartridge 18 is split as described above and removed. With the help of the pusher 9, the graft is advanced to the target site under imaging guidance (FIG. 16). Holding the shaft 14 in place, the thin-wall catheter 7 is withdrawn exposing the leading portion of the graft 1. The basket 15 expands to its original shape, opening the graft 1, and apposing it against the luminal surface of the aorta, thereby securing it. The pusher is removed (FIG. 17). With the shaft 14 of the anchor 10 serving as a guide, a stent 24 is deployed in the graft 1 overlapping its trailing edge, using procedures well known to those skilled in the art, reinforcing apposition of the graft 1 to the luminal surface of the aorta (FIG. 18). The anchor is advanced until the basket 15 is beyond the graft 1 (FIG. 19). Another stent 24′ is placed overlapping the leading edge 3 of the graft (FIG. 20). The thin-wall catheter 7 is advanced until the basket 15 is captured within the lumen of the catheter 7. The anchor 10 is withdrawn (FIG. 21).

(b) Implantation at the Aortic Bifurcation:

An aorto-biiliac bifurcated graft 5a is deployed as described above, ensuring that the entire device lies in the descending aorta. After the first stent is placed in the graft 5a central to the peripheral limb, another graft 1 of appropriate size is implanted in the contralateral iliac artery, such that the leading end of the second graft 1 overlaps the trailing end of corresponding limb of the first graft 5a. The basket 15 in the aorta is advanced out of the graft 5a, and a stent placed across the leading edge 3 of the graft. The anchor 10 is then removed. Another graft 1 is implanted in the ipsilateral iliac artery overlapping the corresponding peripheral limb of the graft 5a in the aorta.

Another option involves the implantation of two aorto-iliac grafts 5b. The first graft is deployed ensuring that its peripheral limb lies in the ipsilateral iliac artery. A stent is placed central to the peripheral limb of the graft. Another stent is placed overlapping the trailing edge of the graft in the iliac artery. The thin-wall catheter 7 is advanced until the basket 15 is captured within the lumen of the catheter 7. The anchor 10 is withdrawn. Via the contralateral femoral artery, another aorto-iliac graft 5b is deployed, ensuring that its peripheral limb protrudes from the aorto-iliac graft 5b already in situ. A stent is placed central to the peripheral limb of the graft. Another stent is placed overlapping the trailing edge of the aorto-iliac graft. The basket 15 is advanced beyond the leading edges of the two grafts in situ. A stent is placed overlapping the leading edges of the two grafts. Another stent is placed overlapping the luminal free edge of the second aorto-iliac graft 5b. The thin-wall catheter 7 is advanced until the basket 15 is captured within the lumen of the catheter. The anchor 10 is withdrawn.

Aorto-biiliac lesions may be alternatively treated by placing two tubular grafts 1 in parallel, with one graft extending into each iliac artery (Sakaguchi S, et. al. Twin-tube endografts for aortic aneurysms: an experimental feasibility study. J Vasc Intervent Radiol 1999; 10:1092-98.)

B. Prosthesis with Helical Support:

The implantation procedures described above are used with a few modifications. The pusher is advanced coaxially over both the shaft 14 of anchor 10 and the part of the internal helical support 2a protruding from the trailing end of graft 1. After the basket 15 expands to its original shape, opening the graft 1 and apposing it against the luminal surface of the organ, the pusher is used to advance the trailing end of internal helical support 2a into the prosthesis, such that it regains its helical configuration.

If the eighth or the ninth embodiments of the invention are to be implanted, the external helical support is deployed in the organ before implantation of the prosthesis is performed, such that the graft is sandwiched between the external and the internal helical supports.

1. A device for transluminal treatment of lesions of tubular organs, that includes:
(a) an implantable prosthesis comprising an uni- or multilamellar tubular member to which is attached multiple linear members whereby said prosthesis is provided with longitudinal support, and,
(b) a deployment instrument for endoluminally implanting said prosthesis comprising:
(i) an outer catheter with an removable inner, coaxial, rigid catheter, and,
(ii) a radially compactable member that is substantially porous on expansion to its non-stressed state.

2. A prosthesis according to claim 1, wherein the linear members attached to the tubular member extend beyond the leading edge, or the trailing edge, or both of the tubular member.

3. A prosthesis according to claim 1, wherein the trailing end of the tubular member is bifurcated into two limbs.

4. A prosthesis according to claim 3, wherein the tubular member has only one limb.

5. A prosthesis according to claim 4, wherein the distal end of the tubular member has only one orifice.

6. A prosthesis according to claim 1, wherein the tubular member is provided with a one or more circumferential slits.

7. A prosthesis according to claim 1, wherein the inner surface of the tubular member is provided with one or more reversibly deformable, helically configured linear members characterized by the leading ends of said helically configured members being attached to the tubular member and the diameter of the described helix being equal to more than the diameter of the tubular member.

8. A prosthesis according to claim 7, wherein the linear longitudinal supporting members are made of a resorbable material.

9. A prosthesis according to claim 7, including one or more reversibly deformable helically configured linear members that are not attached to the tubular member.

10. A method for transluminally treating a disease of a hollow organ, characterized by,
(a) Introduction of the prosthesis and the porous linear member into the organ in a compacted state.
(b) Expansion of the liner porous member such that the prosthesis expands to its nominal diameter and is temporarily secured to the inner surface of the organ.
(c) Implantation of one or more stents in the prosthesis to permanently secure it to the inner surface of the organ.
(d) Advancement of the linear porous member, and implantation of a stent overlapping the leading edge of the prosthesis, or the leading ends of the longitudinal supporting struts.
(d) Removal of the linear porous member.
(e) Deployment of more stents if deemed warranted.

11. The method of claim 9, characterised by the coaxial implantation of multiple prostheses.

12. The method of claim 9, characterized by,
(a) Introduction of the prosthesis and the porous linear member into the organ in a compacted state.
(b) Expansion of the liner porous member such that the prosthesis expands to its nominal diameter and is temporarily secured to the inner surface of the organ.
(c) Release of the trailing end of the helically configured member allowing said member to regain its helical configuration, thereby providing circumferential support to the prosthesis.
(d) Implantation of a first stent in the organ such that it overlaps the trailing edge of the prosthesis, or the trailing ends of the longitudinal supporting struts. (e) Advancement of the linear porous member, and implantation of a second stent overlapping the leading edge of the prosthesis, or the leading ends of the longitudinal supporting struts.
(d) Removal of the linear porous member.
(e) Implantation of more stents in the prosthesis if deemed warranted.

13. The method of claim 9, characterized by the deployment of a helically configured reversibly deformable linear member in the organ prior to implantation of the prosthesis.
